(19) United States
(12) Patent Application Publication
(10) Pub. No.: US 2021/0251852 A1
(43) Pub. Date: Aug. 19, 2021

(54) Aloe Mint Scalp Wipes

(71) Applicant: Tiwona Ross, Park Forest, IL (US)

(72) Inventor: Tiwona Ross, Park Forest, IL (US)

(21) Appl. No.: 17/173,671

(22) Filed: Feb. 11, 2021

Related U.S. Application Data
(60) Provisional application No. 62/976,539, filed on Feb. 14, 2020.

(51) Int. Cl. A61K 8/02; A61K 8/9789; A61K 8/9794; A61Q 19/00

(52) U.S. Cl. CPC A61K 8/0208; A61K 8/9789; A61K 2800/87; A61Q 19/007; A61K 8/9794

(57) ABSTRACT

This present invention relates to scalp nourishing wipes used to prevent dry scalp and to remove any dirt buildup or fungus development that causes itchy scalps. The scalp wipes are infused with a novel aloe and mint based solution. The scalp wipes can be applied to the scalp surface to maintain the moisture level of the skin, which eventually nourishes the skin in single application. The scalp wipes are manufactured using bio-degradable material and is eco-friendly. Further, the scalp wipes of the present invention can be stored in containers that features a sealed lid to prevent the scalp wipes from prematurely drying out.

### CROSS-REFERENCE TO RELATED APPLICATION

The present application claims priority to, and the benefit of, U.S. Provisional Application No. 62/976,539, which was filed on Feb. 14, 2020 and is incorporated herein by reference in its entirety.

### FIELD OF THE INVENTION

The present invention relates generally to the field of wipes. More specifically, the present invention relates to a pre-moisturized wipe that is designed to clean a user's scalp, thereby preventing dirt buildup, fungus development, and more. The novel scalp wipe features an infusion of tea tree oil, anti-septic aloe, and mint that work together to keep the user's scalp both clean and healthy. Regular usage of the novel scalp wipe can prevent buildup of dirt and dead skin cells on a user's scalp. In addition to cleaning the scalp, the present invention scalp wipe also moisturizes and nourishes the scalp. The scalp wipe can be used by the user throughout their active daily routine in order to maintain a clean and moisturized scalp, and may be pre-packaged in containers of various sizes, wherein the containers may be reusable and may include a resealable lid to prevent the wipes from drying out prior to usage. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, the scalp and hair of an individual are protected by sebum, which is a naturally occurring oil that is constantly produced by the sebaceous glands. After sebum is produced by the sebaceous glands, it is excreted through the pores on an individual's scalp, thereby keeping the scalp and hair healthy and moisturized. The sebum oil also forms a barrier on the surface of the scalp to protect it from the environment, including oxidative damage, and further protects the scalp from bacterial infection and other disease-causing microbes.

Frequent hair-washing, especially for those suffering from known skin conditions such as Eczema or Psoriasis, may strip off the naturally occurring oils of the scalp, which may lead to a condition known as “dry scalp.” Dry scalp may occur when the sebaceous glands do not provide enough oil to the scalp for it to be lubricated or moisturized. Similar to dry skin, dry scalp results in itching, flaking or dandruff, irritation and other ailments. The conditions associated with dry scalp can be problematic and may lead to the development of dirt, fungus, bacteria, and unwanted odors on the scalp. Besides the negative effects on an individual's scalp, the lack of the naturally occurring oils may damage the individual's hair as well by causing the hair to dry out and become brittle or broken.

In addition to daily or frequent hair-washing, low-humidity climates, chemical-based hair products, and heat from hair driers may exasperate the dry scalp condition. People with dry skin are more prone to dry scalp. Statistical data shows that millions of people around the world have some degree of dry scalp. Dry scalp, if not treated in time, may lead to dandruff and other ailments, which may require professional cosmetic treatment.

People suffering from dry scalp may treat the condition by applying cosmetic moisturizing lotions and oils, special shampoo, serums, etc. In severe cases of dry scalp, people may need to see a dermatologist for prescribed treatment options. Whether cosmetic or prescribed, many of the products claiming to treat dry scalp may be too expensive for some people to use on an ongoing basis. Additionally, the cosmetic products and medicines may have unwanted side-effects on the individuals as well. Further, cosmetic products such as shampoo, oils, and serums, and prescribed medicines, require the individual to continue using the products in order to maintain their effectiveness.

Therefore, there exists a long felt need in the art for a novel product can keep the user's scalp hydrated and clean. There is also a long felt need in the art for a scalp hydration product that keeps the surface of the user's scalp, and the attached hair, moisturized and hydrated. Additionally, there is a long felt need in the art for a novel scalp wiping product that is easy to use without any negative side-effects, and that has a cleansing effect while preventing dirt buildup, fungus development, and more. Moreover, there is a long felt need in the art for a scalp wipe that protects the user from dry scalp, and its consequences, such as dandruff and other ailments, and that can be applied in a single application. Furthermore, there is a long felt need in the art for a scalp wipe that is portable, affordable and that can be used by a user throughout his or her normal daily routine, thereby keeping the user's scalp clean and moisturized while maintaining the oils on the scalp and preventing dry scalp conditions.

The subject matter disclosed and claimed herein, in one embodiment thereof, comprises a unique scalp wipe that is stacked, interfolded, or integrally interconnected and stored inside a sealable/resealable dispenser. The scalp wipe of the present invention is infused with a solution comprising water, Gluconolactone, Coco glucoside, Tocopherol acetate, Aloe barbadensis, Sodium benzoate, Calcium gluconate, Butyrospermum, Menthol oil, Globulus oil, and Tea tree oil, in a ratio of ingredients that maximizes moisture and nourishment of the skin.

In this manner, the novel scalp wipe of the present invention accomplishes all the forgoing objectives, while providing a safe, convenient and cost-effective solution to moisturize and nourish the user's scalp in a single application. The scalp wipe of the present invention is also infused with ingredients such as tea tree oil, anti-septic aloe, mint, and other substances that have synergistic and therapeutic effect on the user's scalp by moisturizing the scalp and eliminating any buildup of dirt or dead skin cells that can lead to itchy scalp and other ailments. The scalp wipe of the present invention is also user friendly, since the innovative design is less expensive than other alternatives, in addition to being portable as well.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

As used herein regarding the present invention, the term “scalp wipe” refers to a fibrous, paper, cloth sheet, woven or non-woven material that is infused with the unique solution of tea tree oil, anti-septic aloe, mint and other components, which keeps the user's scalp surface hydrated, moisturized, and cleaned. The scalp wipe of the present invention is made from a biodegradable material, and is therefore, both flushable and eco-friendly. The novel scalp wipe can be dispensed through a resealable container, which prevents the scalp wipe from drying out. The resealable packing container allows the scalp wipes to retain their moisture content and effectiveness long after they are first unsealed.

The subject matter disclosed and claimed herein, in one embodiment thereof, comprises a unique and single-application scalp wipe to help a user maintain a hydrated, moisturized, and clean scalp throughout their normal daily routine. The scalp wipe is stacked, interfolded, or integrally interconnected in any convenient quantity, and is further stored in a resealable dispenser. The scalp wipe of the present invention is infused with a novel solution comprising water, Gluconolactone, Coco glucoside, Tocopherol acetate, Aloe barbadensis, Sodium benzoate, Calcium gluconate, Butyrospermum, Menthol oil, Globulus oil, and Tea tree oil, in a ratio of ingredients that maximizes moisture and nourishment of the skin.

In a further embodiment of the present invention, a unique and resealable dispenser for storing the novel scalp wipe is disclosed. The novel resealable wipe dispenser is comprised of a dispenser body that is sized to hold a plurality of the novel scalp wipes, a dispenser lid having a hinged edge and seal, and a dispenser opening sized for easy removal of a single scalp wipe from the wipe dispenser. The air-tight seal of the present invention wipe dispenser prevents the scalp wipes from drying out. The tissue-style container design of the novel wipe dispenser is portable, lightweight and easy to carry, thus enabling users to use and apply the novel scalp wipe as needed.

In a further embodiment of the present invention, a unique solution for impregnating a scalp wipe product is disclosed and comprises water, Gluconolactone, Coco glucoside, Tocopherol acetate, Aloe barbadensis, Sodium benzoate, Calcium gluconate, Butyrospermum, Menthol oil, Globulus oil, and Tea tree oil, in a ratio of ingredients that maximizes moisture and nourishment of the skin. The unique moisturizing and cleansing solution of the present invention is preferably used in conjunction with the novel scalp wipe, but may also be applied directly to the user's scalp by squeezing it from a bottle. A scalp wipe of the present invention or another clothlike material or tissue could then be used to spread the novel moisturizing and cleansing solution into the user's scalp.

In yet another embodiment of the present invention, a method of using a scalp wipe of the present invention is disclosed. The method comprises the steps of opening the lid of a container or dispenser to access a novel scalp wipe that is stored therein, pulling out at least one novel scalp wipe from the container, rubbing the novel scalp wipe on the user's scalp whereby the rubbing action of the scalp wipe will cleanse and moisturize the user's scalp, and finally, disposing of the used scalp wipe.

In a further embodiment of the present invention, a method for producing the novel scalp wipe is disclosed. More specifically, the method comprises the steps of manufacturing a fibrous and porous clothlike material or tissue, preparing an aqueous aloe-mint based solution in a ratio of ingredients that maximizes moisture and nourishment of the skin, infusing the clothlike material or tissue with the aqueous aloe-mint based solution thereby creating the novel scalp wipe, and finally packaging the novel scalp wipes in a stacked or rolled manner inside a sealed container or dispenser that is capable of dispensing a single scalp wipe at a time.

The novel scalp wipe of the present invention is particularly advantageous to the user since the scalp wipe can be used and applied to the scalp conveniently throughout the user's daily routine and is effective in moisturizing and nourishing the scalp skin surface in single application. The scalp wipe of the present invention also removes dirt or dead skin buildup and prevents fungus development, and therefore eliminates itchy scalp and other similar ailments.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the novel scalp wipe of the present invention as per the disclosed design;

FIG. 2 illustrates a diagrammatic composition representation of the unique moisturizing and cleansing solution of the present invention as per the disclosed design;

FIG. 3 illustrates a perspective top view of one potential embodiment of a novel container used for storing and dispensing novel scalp wipes of the present invention as per the disclosed design;

FIG. 4 illustrates a diagrammatic representation of one possible method of applying the novel scalp wipe of the present invention to the scalp of the user as per the disclosed design;

FIG. 5 illustrates a perspective view of a user using one potential embodiment of the novel scalp wipe of the present invention as per the disclosed design; and

FIG. 6 illustrates a perspective view of another potential embodiment of the novel scalp wipe as a circular shaped, interconnected representation of the present invention as per the disclosed design.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a novel product that keeps the scalp of a user hydrated and prevents the user's hair or scalp from drying out. There is also a long felt need in the art for a unique scalp hydration product that keeps the scalp surface moisturized and hydrated. Additionally, there is a long felt need in the art for a novel scalp wipe product of the present invention that can easily be used without any negative side-effects on the user, and that can cleanse the user's scalp by removing dirt and dead skin buildup while preventing fungus development. Moreover, there is a long felt need in the art for a novel scalp wipe that protects the user from dry scalp and its consequences such as dandruff and other ailments, and that can be applied in a single-use application. Furthermore, there is a long felt need in the art for a novel scalp wipe that is portable, cost-effective, affordable, and that can both cleanse and moisturize the user's scalp throughout their daily routine, while maintaining the oils on the scalp thereby preventing dry scalp conditions.

The present invention, in one exemplary embodiment, is a plurality of pre-moistened scalp wipes that are stacked, interfolded, or integrally interconnected and stored in a resealable dispenser. The novel scalp wipe is infused with a solution comprising water, Gluconolactone, Coco glucoside, Tocopherol acetate, Aloe barbadensis, Sodium benzoate, Calcium gluconate, Butyrospermum, Menthol oil, Globulus oil, and Tea tree oil, in a ratio of ingredients that maximizes moisture and nourishment of the skin.

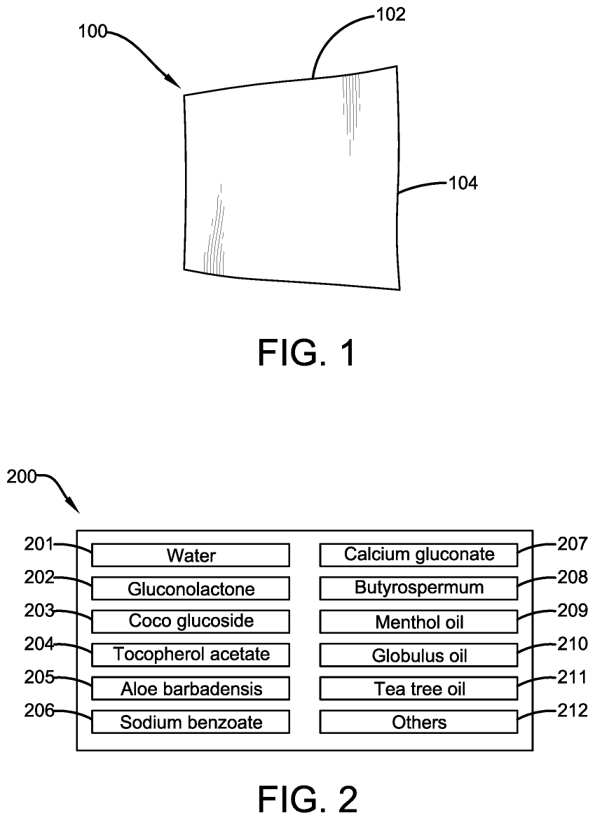

Referring initially to the drawings, FIG. 1 illustrates a perspective view of one potential embodiment of the novel scalp wipe of the present invention as per the disclosed design. As shown in FIG. 1, the scalp wipe 100 is generally rectangular or square in shape, and may have any desired length 102 and width 104. As an example, the scalp wipe 100 is preferably sized to be about 200 mm in length and about 150 mm in width. However, the actual size of the scalp wipe 100 is not so limited, and may be of other dimensions generally ranging from about 100 mm to 350 mm in length and 70 mm to about 250 mm in width. The scalp wipe 100 may also be manufactured in various shapes and sizes based on the user's preferences, and may be packaged as a stack of individual wipes or may include a continuous strip or roll of material having perforations between each scalp wipe 100 for easy tearing and dispensing. The scalp wipe 100 may be manufactured from materials including, but not limited to, cellulosic fibers (cotton, wood pulp, viscose, lyocell), thermoplastic fibers (polyethylene terephthalate, and polypropylene), fibrous, paper, cloth sheet, woven or non-woven material, or others.

FIG. 2 illustrates a diagrammatic representation of the unique infusing solution 200 composition, which is preferably used in conjunction with scalp wipe 100 in FIG. 1 or 600 in FIG. 6 of the present invention. The composition of the infusing solution 200 has several main ingredients, such as tea tree oil, anti-septic aloe, and mint, in a ratio of ingredients that maximizes cleansing and nourishment of the skin, while eliminating any dirt buildup on the scalp surface that may cause itchy scalp and other ailments. Other ingredients may be included as well.

More specifically, the infusing solution 200 comprises water 201, Gluconolactone 202, Coco glucoside 203, Tocopherol acetate 204, Aloe barbadensis 205, Sodium benzoate 206, Calcium gluconate 207, Butyrospermum 208, Menthol Oil 209, Globulus oil 210, Tea tree oil 211 and other ingredients 212 such as, for example, eucalyptus, shea, etc.

Infusing solution 200 preferably includes between 35 percent and 80 percent of water 201 by weight, which makes the solution aqueous. Gluconolactone 202 is a humectant that attracts water and serves to hydrate, soften and soothe the hair and scalp, in addition to creating a barrier on the skin surface, thereby preventing the moisture already present in the skin tissue from evaporating. Coco glucoside 203, a powerful natural cleanser and non-ionic surfactant, is added to infusing solution 200 as a cleansing and conditioning agent. Additionally, Coco glucoside 203 is added to infusing solution 200 to help clean the scalp and hair by combining with the water 201 and cleaning solution to loosen dirt and dead skin and thereby allow it to be washed away.

Infusing solution 200 of the present invention further contains Tocopherol acetate 204, which is a type of Vitamin E belonging to the class of organic chemical compounds known as tocopherols, and is used to prevent hair loss, balance oil production, improve scalp circulation and support a healthy scalp. Tocopherol acetate 204 is also known by a variety of other names including: Alpha tocopheryl acetate (ATA), Vitamin E acetate, A-tocopherol, Alpha tocopherol and D-alpha tocopherol. Infusing solution 200 may also include Aloe barbadensis 205, commonly known as Aloe vera, which is well-known for its antibacterial, antiviral, and antiseptic properties, and for helping to treat skin related issues. Aloe barbadensis 205 is included in the novel infusing solution 200 to treat symptoms of dry scalp including flaking and itching and preferably comprises between 10 and 30% of the solution 200 by weight.

Sodium benzoate 206 is used as a preservative in the novel infusing solution 200. Calcium gluconate 207, a mineral supplement, is included in the infusing solution 200 to treat conditions arising from calcium deficiencies. Butyrospermum 208, also known as Butyrospermum Parkii (Shea) Butter, contains vitamins A, E and F, along with other minerals and nutrients, and is included in the infusing solution 200 to nourish the hair and scalp. Menthol Oil 209 is included in the infusing solution 200 to give it a fresh and pleasant smell, in addition to, providing a tingly sensation and cooling effect to the user's scalp when the novel scalp wipe 100 is used. Additionally, studies have shown that menthol oil 209 increases blood flow of the scalp area and promotes hair growth.

Globulus oil 210, which is extracted from the leaves of Eucalyptus tree (Eucalyptus globulus), is included in the infusing solution 200 since it has natural nourishing properties. Globulus oil 210 stimulates hair follicles, improves hair health, stimulates hair growth, and helps soothe and calm the user's itchy and dry scalp. Finally, Tea tree oil 211, having natural antifungal properties, is included in the infusing solution 200 to treat any fungal scalp conditions such as flaking or dandruff that may be present. Additionally, the tea tree oil 211 makes the hair and scalp healthy, and is preferably between 20 and 30% of the solution by weight.

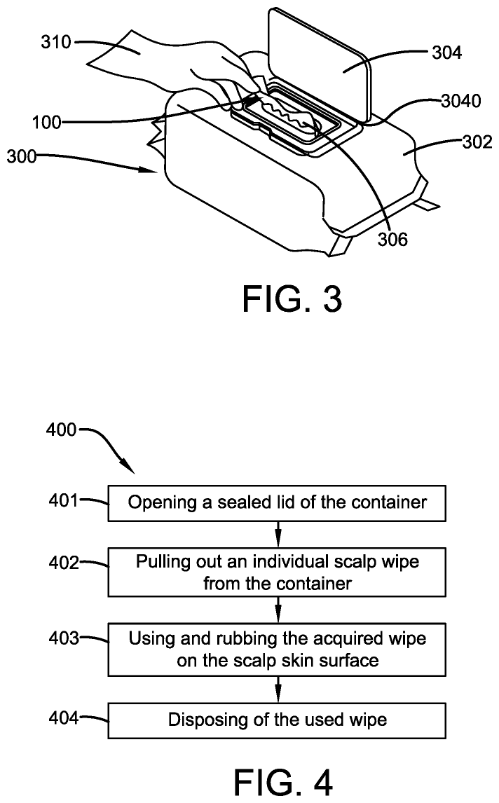

FIG. 3 illustrates a perspective top view of a novel container used for storing and dispensing novel scalp wipes 100 or 600 (as shown in FIGS. 3 and 6, respectively) of the present invention as per the disclosed design. In FIG. 3, an exemplary rectangular-shaped wipe dispenser 300 is shown for storing and dispensing the novel scalp wipe 100 of the present invention. The wipe dispenser 300 features a dispenser body 302 that is preferably manufactured from a durable, but flexible, plastic packaging material such as poly bags and the like. The packaging material for the wipe dispenser 300 forms a complete package for the pre-infused scalp wipes 100. The wipe dispenser 300 of the present invention includes a resealable dispenser lid 304 that is located on the top surface of the dispenser body 302. The dispenser lid 304 is pivotally attached to the dispenser body 302 by hinged edge 3040. The dispenser lid 304 forms an airtight seal with the dispenser boy 302 when it is closed thereby preventing spoilage and drying out of the scalp wipes 100 that are stored inside the novel wipe dispenser 300. The scalp wipes 100 are generally stacked, interfolded, or integrally interconnected within the wipe dispenser 300.

The dispenser lid 304 of wipe dispenser 300 of the present invention may be pivotally attached to the dispenser body 302, or alternatively in another embodiment, made removable and separable from the dispenser body 302. In this embodiment, the dispenser lid 304 may be removed and reattached to the dispenser body 302, and further held in place by snap-fit or frictional fit. A benefit of the removable lid embodiment, allows for the wipe dispenser 300 to be refilled with a fresh supply of scalp wipes 100, thereby making it reusable and extending its use. The dispenser body 302 and the dispenser lid 304 may have logos, customized designs, personalized names, or the like printed on its outer surface, to enhance the appearance of the wipe dispenser 300.

Further, the wipe dispenser 300 may be of any suitable shape and size, including but not limited to: rectangular, cubic, pyramidal, cylindrical, spherical, or any other shape that allows for a stack or roll of scalp wipes 100 to be stored. The wipe dispenser 300 of the present invention is portable, lightweight, and can be easily transported by the user 310 throughout their daily routine. The wipe dispenser 300 is preferably made of plastic material such as polyethylene. In general, other embodiments of the present invention may include wipe dispenser 300 being made from any suitable material, such as water-proof fabric products and other suitable flexible materials. The wipe dispenser 300 may be of any color and design as per the desires and needs of the user 310.

To acquire an individual scalp wipe 100 from the wipe dispenser 300, a user 310 first opens the dispenser lid 304, then pulls out a scalp wipe 100 through the dispenser opening 306 of the wipe dispenser 300, and finally closes the dispenser lid 304. When dispenser lid 304 is closed, it creates an air-tight seal with the dispenser body 302 thereby keeping the remaining scalp wipes in wipe dispenser 300 fresh and moist until they are to be removed by user 310.

FIG. 4 illustrates a diagrammatic representation of a method of applying the novel scalp wipes 100 of the present invention on the scalp of the user as per the disclosed design. The method of application 400 of present invention scalp wipe 100 is easy and convenient for the users. The single application of the scalp wipe 100 allows the user to repeat the method of application 400 whenever and wherever it is necessary. The method of application 400 of the present invention begins with a user opening a sealed lid of the container 401, and then pulling out an individual scalp wipe from the container 402. Next, once the scalp wipe 100 is acquired by the user, the user begins using and rubbing the acquired wipe on the scalp skin surface 403. The user holds the novel scalp wipe 100 with one or both hands and then rubs the scalp wipe 100 on their scalp, such that the infusing solution 200 in the scalp wipe 100 comes in contact with the scalp surface. The user may continue the process of applying the scalp wipe 100 to their scalp for 2-5 minutes as per the desires of the user. Once the scalp wipe 100 is used, the user then ends the method of application 400 by disposing of the used wipe 404. Since scalp wipe 100 is bio-degradable, it may be conveniently flushed or discarded in a trash bin.

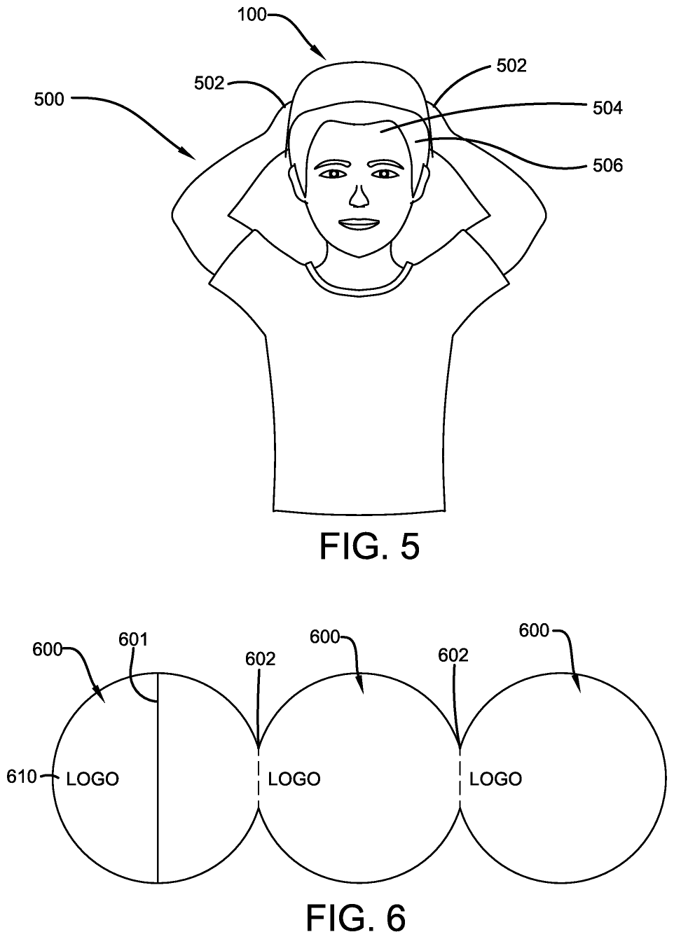

FIG. 5 illustrates a perspective view of a user using the novel scalp wipe 100 of the present invention as per the disclosed design. As shown in FIG. 5, a user 500 uses both the hands 502 to hold the scalp wipe 100 of the present invention over their scalp 504 and hair 506. The user 500 may then gently apply and rub the scalp wipe 100 over his or her scalp surface to impart the advantageous and therapeutic effects of the infusing solution 200 that is contained in the scalp wipe 100 into the scalp 504, and further nourish and treat the conditions of their scalp 504 and hair 506.

FIG. 6 illustrates a perspective view of another potential embodiment of the novel scalp wipe as a circular shaped, interconnected representation of the present invention as per the disclosed design. As shown in FIG. 6, the scalp wipe 600 may be circular in shape with any desired unfolded diameter 601. In an embodiment, the diameter 601 may be preferably in the range of about 3 to 8 inches. The dimensions of the circular scalp wipe 600 can be virtually any size, based on the desires of the users and manufacturers. The scalp wipes 600 may include a continuous strip of material that has perforations 602 between each scalp wipe 600, and may further be arranged in a stack, folded, or rolled into a roll for dispensing. The perforations 602 enables the scalp wipes 600 to be easily torn apart when dispensing from the container. In order to use the novel scalp wipes 600, a user may open the lid of the dispenser, tear away an individual scalp wipe 600 at the perforations 602, and then use the individual scalp wipe 600. Scalp wipes 600 may include a logo 610 or indicia, trademarks, geometric patterns, customizable colors and fonts, embroidery, textures and prints and/or images on the surface.

The container or dispenser used to store the novel scalp wipes of the present invention may include, but are not limited to, individual sachets, flow wrap pouches, chained sachets comprising a tear line between each sachet, and other forms known in the art as suitable for storing nonwoven articles. Additionally, the container may also be manufactured to facilitate the removal of scalp wipes.

The scalp wipes 100, 600 are designed to cleanse the scalp and create optimal conditions for hair growth. The ingredients or composition of the infusing solution 200 in the wipes 100, 600 are chosen to help in the treatment of dry and itchy scalp, and other ailments due to dry scalp conditions. The scalp wipes 100 and 600 of the present invention moisturize and energize the scalp and the hair follicles. The scalp wipes 100 and 600 of the present invention restore the scalp's natural moisture balance and makes sure the scalp remains clean and healthy.

The packaging of the scalp wipes 100 and 600 may include stacking, folding, or rolling. Stored scalp wipes 100 and 600 may be placed into the container individually, or may be attached together, thereby using perforations for easy tearing and removal from the dispenser. The scalp wipe container may be sized to hold any desired quantity of scalp wipes 100 and 600, but may be preferably sized to hold between 1-100 scalp wipes per container.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “scalp wipes”, “scalp nourishing wipes”, and “wipes” are interchangeable and refer to the scalp wipes 100 and 600 of the present invention.

Notwithstanding the forgoing, the scalp wipes 100 and 600 of the present invention can be of any suitable size and configuration as is known in the art without affecting the overall concept of the invention, provided that it accomplishes the above stated objectives. One of ordinary skill in the art will appreciate that the size, configuration and material of the scalp wipes 100 and 600 as shown in the FIGS. are for illustrative purposes only, and that many other sizes and shapes of the scalp wipes 100 and 600 are well within the scope of the present disclosure. Although the dimensions of the scalp wipes 100 and 600 are important design parameters for user convenience, the scalp wipes 100 and 600 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A moisturizing scalp wipe comprising:
a substrate; and
a solution, wherein the substrate is infused with the solution.

2. The moisturizing scalp wipe as recited in claim 1, wherein the solution comprises a quantity of water, a tea tree oil, an anti-septic aloe, and a mint.

3. The moisturizing scalp wipe as recited in claim 2, wherein the solution further comprises a gluconolactone, a coco glucoside and a tocopherol acetate.

4. The moisturizing scalp wipe as recited in claim 3, wherein the solution further comprises an aloe barbadensis, a sodium benzoate and a calcium gluconate.

5. The moisturizing scalp wipe as recited in claim 4, wherein the solution further comprises a butyrospermum and a menthol oil.

6. The moisturizing scalp wipe as recited in claim 5, wherein the solution further comprises a globulus oil.

7. The moisturizing scalp wipe as recited in claim 1, wherein the solution comprises between 35 and 80% by weight of a water.

8. The moisturizing scalp wipe as recited in claim 2, wherein the quantity of water is between 35 and 80% of the solution by weight.

9. The moisturizing scalp wipe as recited in claim 6, wherein the quantity of water is between 35 and 80% of the solution by weight.

10. The moisturizing scalp wipe as recited in claim 2 further comprising an eucalyptus and a shea.

11. The moisturizing scalp wipe as recited in claim 2, wherein the tea tree oil comprises 20 to 30% of the solution by weight and the anti-septic aloe comprises 10 to 30% of the solution by weight.

12. A moisturizing solution for application to a scalp of a user, wherein the moisturizing solution comprises:
a quantity of water;
a tea tree oil;
an anti-septic aloe; and
a mint.

13. The moisturizing solution as recited in claim 12 further comprising a gluconolactone, a coco glucoside and a tocopherol acetate.

14. The moisturizing solution as recited in claim 13 further comprising an aloe barbadensis, a sodium benzoate and a calcium gluconate.

15. The moisturizing solution as recited in claim 14 further comprising a butyrospermum and a menthol oil.

16. The moisturizing solution as recited in claim 15 further comprising a globulus oil.

17. The moisturizing solution as recited in claim 12, wherein the quantity of water is between 35 and 80% of the moisturizing solution by weight.

18. The moisturizing solution as recited in claim 16, wherein the quantity of water is between 35 and 80% of the moisturizing solution by weight.

19. A scalp moisturizing system comprising:
a dispenser;
a plurality of substrates positioned in the dispenser; and
a solution, wherein each of the plurality of substrates is infused with the solution and further wherein the solution comprises a quantity of water, a tea tree oil, an anti-septic aloe, a mint, a gluconolactone, a coco glucoside, a tocopherol acetate, an aloe barbadensis, a sodium benzoate, a calcium gluconate, a butyrospermum, a menthol oil and a globulus oil.

20. The scalp moisturizing system as recited in claim 19, wherein the quantity of water is between 35 and 80% of the solution by weight.
